(19) United States
(12) Patent Application Publication
(10) Pub. No.: US 2002/0051786 A1
(43) Pub. Date: May 2, 2002

(54) Modulators of p85 expression

(76) Inventors: C. Ronald Kahn, West Newton, MA (US); Kohjirro Ueki, Boston, MA (US); Frank Mauvais-Jarvis, Paris (FR); David Fruman, Brookline, MA (US); Lewis Cantley, Cambridge, MA (US)

Correspondence Address: LOUIS MYERS, Fish & Richardson P.C., 225 Franklin Street, Boston, MA, 02110-2804, US

(21) Appl. No.: 09/887,487

(22) Filed: Jun. 22, 2001

Related U.S. Application Data
(60) Provisional application No. 60/214,222, filed on Jun. 23, 2000.

(51) Int. Cl. A61K 39/395

(52) U.S. Cl. 424/146.1; 424/145.1

(57) ABSTRACT

Methods of treating a subject having an insulin-related disorder, e.g., diabetes. The methods include reducing the amount of p85 P13K regulatory subunit isoform in a cell of the subject.

### RELATED APPLICATIONS

This application claims the benefit of U.S. Provisional Application Ser. No. 60/214,222, filed on Jun. 23, 2000, which is incorporated herein by reference in its entirety.

### FEDERALLY SPONSORED RESEARCH OR DEVELOPMENT

The U.S. Government may have certain rights in this invention pursuant to Grant No. RO1 GM41890 awarded by the National Institutes of Health.

### FIELD OF THE INVENTION

The invention relates to methods of diagnosing and treating insulin-related disorders.

### BACKGROUND OF THE INVENTION

The treatment of insulin resistant states and type 2 diabetes remains problematic. Basic phatophysiologic studies have suggested that a main component, perhaps the earliest component, in the development of type 2 diabetes is insulin resistance. Among currently available agents for the treatment of type 2 diabetes, thiazolidiones are directed to improving insulin sensitivity. This class of agents works through the mechanism of increasing the expression of some insulin sensitive genes, in particular, glucose transporter genes. The biguadides, such as Metformin, also have some effects on insulin-sensitive tissues, especially the liver, but their mechanism of action remains unknown. The treatment of patients having type 2 diabetes frequently requires multiple agents, and even with these agents, the control of blood glucose is often poor. In addition to type 2 diabetes, insulin resistance is common to a number of other conditions, such as obesity, hypertension, polycystic ovarian disease, and various hypolipidemias.

### SUMMARY

In general, the invention features a method of treating a subject having an insulin-related disorder. An insulin-related disorder as defined herein includes diabetes, e.g., type 2 diabetes, and atypical insulin resistant states. The method includes: optionally identifying a subject in need of treatment for an insulin-related disorder, and altering, e.g., reducing, the expression, and/or amount, and/or activity of p85, e.g., p85a or p85P, in a cell or tissue of the subject, e.g., a liver, fat (e.g., brown adipose), heart, or skeletal muscle cell or tissue.

In a preferred embodiment, the expression and/or amount and/or activity of all isoforms of p85α (p85α, p50α, and p55α) are reduced. In another preferred embodiment, the amount, and/or expression and/or activity of p85β is reduced. Preferably, reducing the expression and/or activity of a p85 isoform, e.g., a p85α or p85β isoform monomer, alters the interaction of the p85α or p85β monomer with p110 and/or insulin receptor substrate (IRS), in a cell or tissue of the subject. While not wishing to be bound by theory, it is believed that reducing the expression and/or activity of p85 monomers in a cell or tissue can increase the association of p85-p110 dimers with an IRS, e.g., IRS-1, thereby increasing insulin signaling and glucose uptake.

As used herein, “altering” can mean increasing or reducing the amount of p85, e.g., increasing or decreasing the amount of p85α or β; increasing or reducing the level of p85α, or β mRNA and/or p85α or β protein expression; or increasing or reducing the activity of p85α or β protein. Preferably, “altering” means reducing. A reduction in the availability of p85, e.g., p85α or β, can result in improved insulin sensitivity and glucose tolerance. Preferably, a reduction of the amount, expression, or activity of a p85 isoform is a decrease of less than 100%. Preferably, a p85 isoform is reduced between 10% and 95%, more preferably between 20% and 80%, even more preferably between 40% and 60%, e.g., 50% as compared to a control.

As used herein, “p85” or “p85 isoform” is a p85α or p85β isoform. A p85α isoform can be any of: p85α, p50α, or p55α.

Accordingly, in one aspect, the invention features a method of treating a subject, e.g., a human or a non-human animal, having an insulin-related disorder (e.g., diabetes; hyperglycemia; obesity; hypertension; polycystic ovarian disease; or hypolipidemia). The method includes reducing the level of p85, e.g., p85α or p85β, in a cell, e.g., a liver, fat, heart, or skeletal muscle cell, of the subject.

In a preferred embodiment, the insulin related disorder is diabetes, preferably Type 2 diabetes; obesity; hypertension; polycystic ovarian disease; or hypolipidemia.

In a preferred embodiment, the level of expression or activity of p85α is reduced. Preferably, reducing the level of p85α includes reducing the level of all isoforms of p85α.

In another preferred embodiment, the level of expression of p85β is reduced.

In a preferred embodiment, the level of expression of p85α and p85β are both reduced.

In a preferred embodiment, the subject is an experimental animal, e.g., a mouse model of insulin resistance and/or hyperglycemia, e.g., a mouse heterozygous for a knock out of the insulin receptor (IR), a mouse heterozygous for a knockout of IRS-1, or a mouse heterozygous for a knockout of IR and IRS-1.

In a preferred embodiment, the subject is a human.

In a preferred embodiment, reducing the level of active p85, e.g., p85α or p85β, includes administering an anti-p85α or anti-p85β antibody or a small molecule that reduces the level of active p85α or p85β. In a preferred embodiment, the anti-p85 antibody or small molecule interacts, e.g., binds, to an SH2 or SH3 domain of p85.

In a preferred embodiment, the cell is a liver, heart, fat (e.g., brown fat), or skeletal muscle cell.

In a preferred embodiment, the method includes: decreasing the amount of active p85, e.g., p85α or p85β, in a cell, e.g., a liver cell, heart cell, fat cell, or skeletal muscle cell, of a subject, e.g., by administering a compound which inhibits expression of p85, e.g., p85α or p85β, or which interacts with, e.g., binds, to p85, e.g., p85α or p85β, to thereby inhibit or sequester the p85 isoform. In a preferred embodiment, the compound interacts, e.g., binds, to an SH2 or SH3 domain of p85.

“Active p85” refers to p85, e.g., p85α or p85β, in a cell available for interacting with p110 as part of the PI3K signaling cascade. For example, active p85 is a p85 monomer. The amount of active p85 can be decreased by either decreasing the total amount of p85 in a cell and/or by inhibiting the functional activity of p85, e.g., the ability to bind an IRS, that is present in a cell. In preferred embodiments, the active levels of p50α and/or p55α are also decreased.

Compounds which bind, and preferably thereby inhibit or sequester, p85, e.g., p85α or p85β, can be used to decrease p85, e.g., p85α or p85β. Such compounds can include: anti-p85 antibodies, soluble fragments of p85 ligands, e.g., p110, small molecules, and random peptides selected, e.g., selected in a phage library, for the ability to bind to p85.

Peptides are examples of compounds which can bind, inhibit and/or sequester p85, e.g., p85α or p85β. For example, peptide fragments of p110 or small peptides that have been selected on the basis of binding p85 can be used. These can be selected in phage display or by similar methods. Such peptides are preferably at least four, more preferably at least six or ten amino acid residues in length. They are preferably less than 100, more preferably less than 50 and most preferably less than 30 amino acids in length. Preferably, the peptide inhibits the ability of p85α to interact with, e.g., bind to, a p85α ligand, e.g., p110. In one embodiment, the peptide binds to an active domain of p85α, e.g., an SH2 domain, an SH3 domain, a Rho-GAP homology domain, and/or a polyproline domain.

Small molecules can also be used. “Small molecules”, as used herein, refers to a non-peptide compound which is preferably of less than 5,000, more preferably less than 2,500, most preferably less than 1,500 in molecular weight. Preferably, a small molecule binds to a p85, e.g., p85α or p85β, and inhibits at least one of its wild-type functions, e.g., inhibits an interaction with p110 or an IRS, e.g., IRS-1. Preferably, the interaction between the small molecule and p85 results in increased insulin sensitivity. In one embodiment, the small molecule binds to an active domain of p85, e.g., an SH2 domain, an SH3 domain, a Rho-GAP homology domain, and/or a polyproline domain.

The level of free or active p85, e.g., p85α or p85β, can also be reduced by administration of a nucleotide sequence which binds to and inhibits p85 expression, e.g., a p85 antisense molecule. In preferred embodiments, the p85 antisense molecule is delivered by, e.g., gene or cell therapy. In other embodiments, the p85 antisense molecules are delivered by the administration of the oligonucleotides.

The level of p85, e.g., p85α or p85β, expression can also be inhibited by decreasing the level of expression of an endogenous p85 gene, e.g., by decreasing transcription of the p85 gene. In a preferred embodiment, transcription of the p85 gene can be decreased by: altering the regulatory sequences of the endogenous p85 gene, e.g., by the addition of a negative regulatory sequence (such as a DNA-binding site for a transcriptional repressor).

The level of p85, e.g., p85α or p85β, expression can also be inhibited by administering one or more anti-p85, e.g., anti-p85α or anti-p85β, antibodies. An anti-p85 antibody can be a polyclonal or a monoclonal antibody. In other embodiments, the antibody can be recombinantly produced, e.g., produced by phage display or by combinatorial methods. In one embodiment, the peptide binds to an active domain of p85, e.g., an SH2 domain, an SH3 domain, a Rho-GAP homology domain, and/or a polyproline domain.

In another preferred embodiment, the invention further includes: increasing the level of p85-p110 dimer in a cell of the subject. The level of p85-p110 dimer can be increased by, e.g., providing a nucleic acid encoding p110 or a functional fragment or analog thereof and/or a p110 protein or functional fragment or analog thereof. A nucleic acid encoding p110 or a functional fragment or analog thereof can be delivered, e.g., by gene or cell therapy. Alternatively, the level of p110 can be increased by providing a substance that increases transcription of p110. In a preferred embodiment, transcription of p110 is increased by: altering the regulatory sequences of the endogenous p110 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the p110 gene to be transcribed more efficiently. In another preferred embodiment, the level of p110 can be increased by, e.g., providing an agent which increases the level of p110, e.g., a small molecule which binds to the promoter region of p110.

In preferred embodiments, the subject has exhibited at least one indication of an insulinrelated disorder, e.g., insulin resistance, hyperglycemia, prior to receiving a treatment provided herein. In one embodiment, the subject has type 2 diabetes.

In other embodiments, a treatment described herein is provided to a subject in the absence of the subject having exhibited symptoms of an insulin-related disorder. In one embodiment, the subject is thought to be at risk for an insulin-related disorder, e.g., insulin resistance.

In another aspect, the invention provides a method of determining if a subject is at risk for a disorder, e.g., an insulin-related disorder, e.g., a disorder related to a lesion in or the misexpression of the gene which encodes a p85 isoform.

Such disorders include, e.g., a disorder associated with the misexpression of p85; a disorder associated with glucose uptake; and/or a disorder associated with insulin sensitivity such as type 2 diabetes.

In a preferred embodiment, the method includes evaluating the expression of p85 to determine if the subject is at risk, to thereby determine if a subject is at risk.

In a preferred embodiment, the method includes one or more of the following:

detecting, in a tissue of the subject, the presence or absence of a mutation which affects the expression of a p85 gene, or detecting the presence or absence of a mutation in a region which controls the expression of the gene, e.g., a mutation in the 5′ control region;

detecting, in a tissue of the subject, the presence or absence of a mutation which alters the structure or expression of a p85 gene;

detecting, in a tissue of the subject, the misexpression of a p85 gene, at the mRNA level, e.g., detecting a non-wild type level of a mRNA, e.g., wherein increased levels of p85a mRNA is associated with decreased insulin sensitivity, e.g., is indicative of a risk of type 2 diabetes;

detecting, in a tissue of the subject, the misexpression of the p85 gene, at the protein level, e.g., detecting a non-wild type level of a p85 polypeptide, wherein increased levels of p85 protein is associated with decreased insulin sensitivity, e.g., is indicative of a risk of type 2 diabetes.

In preferred embodiments the method includes: ascertaining the existence of at least one of: a deletion of one or more nucleotides from the p85 gene; an insertion of one or more nucleotides into the gene; a point mutation, e.g., a substitution of one or more nucleotides of the gene; a gross chromosomal rearrangement of the gene, e.g., a translocation, inversion, or deletion.

For example, detecting the genetic lesion can include: (i) providing a probe/primer, e.g., a labeled probe/primer, which includes a region of nucleotide sequence which hybridizes to a sense or antisense sequence from the p85 gene, or naturally occurring mutants thereof, or to the 5′ or 3′ flanking sequences naturally associated with the p85 gene; (ii) exposing the probe/primer to nucleic acid of the tissue; and detecting, by hybridization, e.g., in situ hybridization, of the probe/primer to the nucleic acid, the presence or absence of the genetic lesion.

In a preferred embodiment, detecting the misexpression includes ascertaining the existence of at least one of: an alteration in the level of a messenger RNA transcript of the p85 gene, e.g., as compared to levels in a subject not at risk for an insulin related disorder; the presence of a non-wild type splicing pattern of a messenger RNA transcript of the gene; or a non-wild type level of the p85 protein e.g., as compared to levels in a subject not at risk for an insulin related disorder.

Methods of the invention can be used prenatally or to determine if a subject's offspring will be at risk for a disorder.

In a preferred embodiment, the method includes determining the structure of a p85 gene, an abnormal structure being indicative of risk for the disorder.

In a preferred embodiment, the method includes contacting a sample from the subject with an antibody to the p85 protein or a nucleic acid, which hybridizes specifically with a portion of the gene.

In another aspect, the invention features a method of screening for a compound that binds a p85, e.g., a p85α or β isoform monomer, e.g., p85α, p55α, p50α, or p85β. The method includes: a) providing a test agent; b) contacting the test agent with a p85 isoform described herein; and c) determining whether the test agent binds to the p85 isoform.

In a preferred embodiment, the method further includes administering the test agent to an experimental model, e.g., a mouse model for insulin resistance described herein.

In a preferred embodiment, the method further includes evaluating the ability of the test agent to alter the interaction of the p85 isoform with p110 or IRS-1.

In a preferred embodiment, the method further includes evaluating the ability of the test agent to alter AKT activity, PIP3 formation, or phosphorylation of Bad, FKHR or CREB.

In a preferred embodiment, the method further includes evaluating the ability of the test agent to bind at least 2, preferably all, p85α isoforms.

In a preferred embodiment, the test agent is selected from the group of: a peptide, an antibody, a small molecule.

In a preferred embodiment, contacting the test agent with a p85 isoform includes contacting the test agent with a cell expressing a p85 isoform.

In another aspect, the invention features a method of identifying a compound for treatment of an insulin related disorder. The method includes: a) providing a test agent; b) administering the test agent to a cell, tissue, or experimental animal; and c) evaluating the ability of the test agent to reduce the amount and/or expression and/or activity of a p85 isoform, e.g., a p85α isoform (e.g., p85α, p50α, or p55α), or a p85β isoform. An agent that reduces the activity of a p85 isoform is identified as an agent for the treatment of an insulin related disorder.

In a preferred embodiment, the test agent is evaluated for its ability to reduce the activity of at least 2, preferably all, p85α isoforms.

In a preferred embodiment, the ability of the agent to reduce the activity of a p85 isoform in the cell, tissue, or experimental animal is evaluated by evaluating P13K or p110 activity in the cell, tissue, or experimental animal, e.g., by comparing PI3K or p110 activity prior to and after administration.

In a preferred embodiment, the ability of the agent to reduce the activity of a p85 isoform in the cell, tissue, or experimental animal is evaluated by determining the ability of the agent to affect insulin sensitivity in the cell, tissue, or experimental animal.

In a preferred embodiment, the cell or tissue is a fat, liver, heart, or skeletal muscle cell or tissue.

In a preferred embodiment, the experimental animal is an animal model (e.g., a rodent model) for insulin resistance, e.g., IR heterozygotes, IRS-1 heterozygotes, or IR/IRS-1 double heterozygotes.

In a preferred embodiment, the agent is selected from the group consisting of a peptide, an antibody and a small molecule.

In a preferred embodiment, the insulin related disorder is diabetes or hyperglycemia.

In another aspect, the invention features a method of analyzing a treatment for its effect, e.g., for its effect on insulin metabolism, e.g., insulin sensitivity or glucose uptake, in a subject.

The method includes providing an animal or a cell, in which the ratio of p85α to one or more of p110, p85β, p55γ, or IRS has been altered. Preferably, the ratio of p85α to any of p110, p85β, p55γ, and IRS has been decreased. In a preferred embodiment, the subject is a genetically modified animal having a genetic lesion, for example a knockout, at the gene which encodes p85α. This animal may be useful to compare the effectiveness of a treatment in a wild type animal, wherein the treatment is designed to reduce the amount of active p85α.

A treatment, e.g., a compound administered to the subject, can be evaluated for its effect on insulin metabolism, for example, insulin sensitivity.

In another embodiment, the subject is a transgenic animal, e.g., a transgenic rodent, e.g., mouse, having a transgene, for example a transgene which encodes p85β.

In another embodiment, the subject is a transgenic animal, e.g., a transgenic rodent, e.g., mouse, having a transgene, for example, a transgene which encodes p85α. In this embodiment, the transgenic mouse may be useful as a model for decreased insulin sensitivity, e.g., type 2 diabetes.

“Misexpression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### DESCRIPTION OF THE DRAWINGS

FIG. 1 shows that Pik3r1 mice have lower glucose and insulin concentrations than wild type. a) Fasting glucose (top left) and insulin (bottom left) concentrations as well as random fed glucose (top right) and insulin (bottom right) concentrations were determined by tail bleeding in 2-3 week Pik3r1^{-/-} and wild type (WT) mice. Values for glucose levels represent the mean +s.e.m. of n=6-17 mice per genotype. **P<0.01 Pik3r1^{-/-} versus WT. Insulin concentrations determined by ELISA are shown as a scatter plot. b) i.p. GTT (2 g/kg) was performed on overnight fasted 3-week Pik3r1^{-/-} and WT mice. Glucose concentrations (left) were measured by tail bleeding at the indicated time points. Insulin concentrations (right) were determined 60 minutes after glucose injection by ELISA. Values represent the mean +s.e.m. of n=6 mice per genotype. *P<0.05, **P<0.0l Pik3r1^{-/-} versus WT.

### DETAILED DESCRIPTION

The invention provides methods of modulating the expression of class 1_{A}PI3K regulatory subunit genes or inhibiting the function of various domains of class 1_{A}PI3 K regulatory subunit molecules as a treatment for insulin resistance and type 2 diabetes.

Phosphoinositide 3-kinases (PI3Ks) are enzymes that phosphorylate the D-3 position of phospholipids containing an inositol headgroup (phosphoinositides). PI3Ks are involved in many cellular responses triggered by external stimuli. For example, insulin-dependent glucose uptake is thought to require PI3K activation. Several classes of P13Ks exist in mammalian cells. Class I_{A}PI3Ks are heterodimers of a catalytic subunit of about 110 kDa (p110) and a regulatory subunit, usually of about 85 kDa (p85).

Three genes encoding regulatory subunits have been identified in mammals. The gene encoding p85α (Pik3r1) also encodes two smaller variants, p55α and p50α. p85β is derived from a second gene, and p55γ is derived from a third gene. p85α and p85β each contain two Src homology 2 (SH2) domains and one SH3 domain. p55α, p50α, and p55γ lack the SH3 domain and contain unique amino acid sequences at the amino terminus.

The role of P13K in insulin signaling is as follows. The insulin receptor tyrosine kinase is activated by binding of insulin to the extracellular region of it receptor. The activated tyrosine kinase phosphorylates IRS proteins on numerous phosphotyrosine (pTyr) residues. Some of these are specific binding sites for the SH2 domains of class I_{A} regulatory subunits. Association of PI3K with IRS proteins increases the lipid kinase activity of the p110 subunit and brings it into proximity with substrates at the membrane. The lipid products act as second messengers to recruit other signaling proteins to the membrane. This signaling eventually leads to glucose uptake by the cell. The importance of PI3K in this signaling process is supported by two general types of experiments. First, compounds that inhibit p110 kinase activity (e.g., wortmannin, Ly294002) block insulin-mediated glucose transport in cultured cells. Second, expression of constitutively active forms of PI3K can stimulate glucose transport and dominant negative forms can inhibit glucose transport.

p85α Knockout Mice

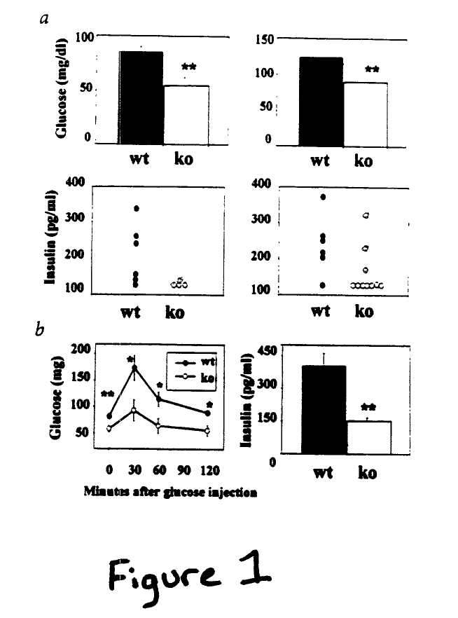

Deletion of class I_{A} regulatory subunits by gene targeting was predicted to result in insulin resistance and possibly diabetes, as is seen in mice lacking the insulin receptor or certain IRS proteins. To test this, mice were created which lacked the Pik3r1 gene, and thus lacked all three p85α isoforms encoded by the Pik3r1 gene (p85α, p55α, and p50α). Surprisingly, the mice were hypoglycemic, despite lower serum insulin levels in the fed state (FIG. 1). Fasted animals show enhanced glucose disposal in a glucose tolerance test, while maintaining lower insulin levels. Biochemical studies of insulin-stimulated liver and muscle revealed that loss of Pik3r1 expression in homozygous tissues was associated with an 80-90% reduction in total class I_{A} Pi3k activity as detected in pan-p85 immunoprecipitates but there was normal activation of the PI3K downstream target Akt/PKB in Pik3r1 −/− mice, suggesting that the output of PI3K signaling is unimpaired in vivo, despite disruption of the Pik3r1 gene. The expression of the genes encoding p85β and p55γ regulatory isoforms was increased in the liver and muscle of Pik3r1−/− mice, thus providing a possible compensatory mechanism.

Insulin sensitivity could not be tested directly in Pik3r1^{-/-} mice because homozygous mice died before adulthood. To determine the cause of perinatal lethality in these mice, tissue samples were tained with haematoxylin and eosin. Many animals had livers with areas of necrosis that was confined to the hepatocytes and did not affect the hematopoietic cells. The absence of nuclei or nuclear fragments from most of the hepatocytes was consistent with death by necrosis, not apoptosis. The hearts of two animals showed round nodules that appeared to be calcified and two animals had extensive necrosis of brown fat cells, suggesting that necrosis was not confined to the liver. Another histological abnormality of Pik3r1^{-/-} mice was the presence of enlarged skeletal muscle fibers.

Pik3r1 +/− mice were viable, exhibiting reduced expression of Pik3r1 gene products and had some increase in p85β expression. These mice demonstrated hypoglycemia, although the hypoglycemia was milder than that detected in the Pik3r1 −/− mice. The Pik3r1 +/− mice exhibited improved glucose tolerance relative to their wild-type littermates. Insulin tolerance tests showed a significant increase in insulin sensitivity in Pik3r1 +/− mice.

The presence of a single disrupted allele of Pik3r1 (Pik3r1 +/−) improved insulin sensitivity in three separate models of insulin resistance in mice: (1) Insulin receptor heterozygotes (IR +/−), insulin receptor substrate-1 heterozygotes (IRS-1 +/−), and IR/IRS-1 double heterozygotes. In the IR/IRS-1 double heterozygotes, overt diabetes was prevented in about 50% of the IR/IRS-1/Pik3r1 heterozygotes.

In order to determine the basis for these phenotypes, wild-type cells were compared to cells with heterozygous or homozygous disruption of the p85α gene. It was found that in wild-type cells, the regulatory p85 subunit of PI3-kinase is more abundant than the p110 catalytic subunit. This leads to competition between p85 monomer and p85-p110 dimer for binding to phosphorylated proteins, e.g., phosphorylated IRS proteins, and ineffective signaling. In cells with heterozygous disruption of the p85α gene, there is a preferential decrease in p85 monomer that competes with p85-p110 dimer for binding insulin receptor substrate (IRS) proteins, and an increase in the ratio of p85-p110 dimer to p85 monomer, thereby improving the stoichiometery of p85/p110/IRS complex and efficiency of signaling. Thus, these cells exhibit normal PI 3-kinase activity and increased PIP₃ formation in response to insulin-like growth factor-1 (IGF-1) stimulation despite a 50% reduction on p85α. The increased PIPK3 formation seems to be caused, at least in part, by an attenuation of lipid phosphates PTEN activity, which occurs independent of PI 3-kinase activity. This leads to an increase in Akt activity, phosphorylation of Bad, FKHR and CREB, and enhanced cell survival following serum starvation. Complete disruption of p85α, on the other hand, markedly decreased the level of p85-p110 dimer, resulting in a reduction of PI 3-kinase activity, PIPˆ 3 levels, AKT activity and phosphorylation of Bad, FKHR and CREB. These cells therefore exhibit high levels of apoptosis following serum starvation and are resistant to IGF-1's anti-apoptotic effects.

Together, these data indicate that normal cells have an imbalance of catalytic and regulatory subunits of PI 3-kinase, and that reduction of p85α can improve IGF-1 and insulin signaling, through an increase in net PIP, production. Thus, reducing p85a levels represents a novel therapeutic target for enhancing insulin/IGF-1 signaling, prolongation of cell survival and protection against apoptosis.

p85β Knockout Mice

Disruption of p85β, which represents 10-20% of total p85 regulatory subunits, also results in hypoglycemia and improved insulin sensitivity, albeit to a lesser extent that the p85α(+/−) mice, presumably due to a mechanism similar to that in the p85α(+/−) mice. However, different from the p85α(+/−) mice, the p85 p(+/−) mice exhibit unregulated Akt activity and phosphorylation of IRS-2 in muscle and brown adipocytes. This indicates that the relative contribution of p85α and p85β is different in each tissue, and that the p85β may have a specific role in insulin signaling, particularly in muscle and brown adipocytes. Because reducing p85β protein improved insulin sensitivity, the p85β regulatory subunits also represent a novel therapeutic target in the treatment of insulin resistant states, e.g., type 2 diabetes and other conditions described herein.

The studies of the heterozygous and homozygous mice described herein suggest that altering the balance of expression of P13K regulatory isoforms can influence the sensitivity of insulin signaling in vivo. Without wanting to be bound by theory, it is thought that class I_{A} regulatory proteins, e.g., p85α and/or β, are normally in excess in insulin-responsive tissues. Thus, free regulatory subunits would compete for binding to phosphorylated IRS proteins with heterodimeric class I_{A} complexes and possibly with other SH2 domain-containing signaling proteins. Reducing the abundance (preferably reducing less than 100%), of the free regulatory subunits, e.g., p85α isoforms or p85β, can allow more efficient activation of P13K and possibly other targets. Thus, compounds found to reduce the amount, expression, or activity of individual p85 isoforms are useful in the treatment of insulin-resistance syndromes such as type 2 diabetes.

The knockout mice described herein can be used in various ways. For example, the mice can be used as a benchmark with which to compare drugs that regulate PI3K subunit expression. For example, a drug that results in reduced p85 expression, increased numbers of p85-p110 heterodimers, increased localization of p85-p110 heterodimers to active sites on the cell membrane, or increased activation of PI3K, can effect an increase insulin sensitivity in diabetic subjects.

The knockout mice can also be used to develop drugs that modulate function of subdomains of PI3K regulatory isoforms, such as SH2, SH3, Rho-GAP homology and polyproline domains.

A number of methods could be employed to alter the expression of p85 or the functional interaction between p85 and p110 and/or IRS. These methods include, for example the use of antibodies, or antisense or ribozymes as described herein. Other approaches include, e.g., the use of small molecules which regulate gene expression at the transcriptional or post-transcriptional level. For example, such small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Assays for p85 Activity

p85 activity can be assayed by a number of methods known in the art. For example, the amount of p85α or p85β present in a sample or subject can be assayed by standard immunoprecipitation experiments using known p85α or p85β antibodies that are commercially available. In addition, PI3K assays, which are routine in the art, may be used to determine p85, e.g., p85α or p85β, activity. An exemplary PI3K assay is described, e.g., in Kelly et al. (1993) J. Biol. Chem. 268: 4391-4398, the contents of which are hereby incorporated by reference in their entirety.

Antisense Nucleic Acid Molecules and Ribozymes

The methods described herein can comprise modulating, e.g., inhibiting, p85 activity by antisense techniques. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire p85 coding strand, or to only a portion thereof (e.g., the coding region of a p85). In another embodiment, the antisense nucleic acid molecule is antisense to a “noncoding region” of the coding strand of a nucleotide sequence encoding a p85 (e.g., the 5′ and 3′ untranslated regions).

An antisense nucleic acid can be designed such that it is complementary to the entire coding region of p85 mRNA, but more preferably is an oligonucleotide that is antisense to only a portion of the coding or noncoding region of p85 mRNA. For example, the antisense oligonucleotide can be complementary to the region surrounding the translation start site of p85 mRNA, e.g., between the −10 and +10 regions of the target gene nucleotide sequence. An antisense oligonucleotide can be, for example, about 7, 10, 15, 20, 25, 30, 35, 40, 45, 50, 55, 60, 65, 70, 75, 80, or more nucleotides in length.

Preferably, an antisense nucleic acid complementary to the p85α gene inhibits the expression of the p85α, p50α, and p55α isoforms encoded by the p85α gene.

An antisense nucleic acid of the invention can be constructed using chemical synthesis and enzymatic ligation reactions with procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

The antisense nucleic acid molecules of the invention are typically administered to a subject (e.g., by direct injection at a tissue site), or generated in situ such that they hybridize with or bind to cellular mRNA and/or genomic DNA encoding a p85 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong polymerase II or polymerase III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid of the invention is a ribozyme. A ribozyme having specificity for a p85-encoding nucleic acid can include one or more sequences complementary to the nucleotide sequence of a P85 cDNA, and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a p85-encoding mRNA. See, e.g., Cech et al. U.S. Pat. No. 4,987,071; and Cech et al. U.S. Pat. No. 5,116,742. Alternatively, p85 mRNA can be used to select a catalytic RNA having a specific ribonuclease activity from a pool of RNA molecules. See, e.g., Bartel and Szostak (1993) Science 261:1411-1418.

p85 gene expression can be inhibited by targeting nucleotide sequences complementary to the regulatory region of a p85 gene (e.g., the p85 promoter and/or enhancers) to form triple helical structures that prevent transcription of a p85 gene in target cells. See generally, Helene, C. (1991) Anticancer Drug Des. 6(6):569-84; Helene, C. et al. (1992) Ann. N.Y Acad. Sci. 660:27-36; and Maher, L. J. (1992) Bioassays 14(12):807-15. The potential sequences that can be targeted for triple helix formation can be increased by creating a “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Transgenic Animals

The invention provides non-human transgenic animals. As used herein, a “transgenic animal” is a non-human animal, preferably a mammal, more preferably a rodent such as a rat or mouse, in which one or more of the cells of the animal includes a transgene. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., in a “knockout” animal, reduce or eliminate expression.

Preferred transgenic animals of the invention are animals, e.g., mice, that are models of insulin resistance. Such animals can have more than one transgene, for example, a mouse may have two or more transgenes selected from the group of an insulin receptor (IR) transgene, an insulin receptor substrate (IRS) transgene, and a Pik3r1 transgene. A Nod mouse or another known mouse model for diabetes can also be used as a background to make a transgenic animal of the invention.

Antibodies

In another aspect, the invention features antibodies which inhibit a p85 isoform, e.g., p85α, p85β, p50α, or p55α, to thereby treat a subject having an insulin related disorder, e.g., diabetes.

An anti-p85 antibody or fragment thereof can be used to bind a p85, and thereby reduce p85 activity. Anti-p85 antibodies can be administered such that they interact with p85 protein locally at the site of alteration but do not inhibit p85 expression generally in the cell.

The p85 protein, or a portion or fragment thereof, can be used as an immunogen to generate antibodies that bind p85 using standard techniques for polyclonal and monoclonal antibody preparation. The full-length p85 can be used or, alternatively, antigenic peptide fragments of a p85 isoform can be used as immunogens, e.g., a p85 SH2 or SH3 domain or a p55α or p50α unique domain can be used as an immunogen. In a preferred embodiment, the antibody binds to a p85 SH2 or SH3 domain, or a portion thereof.

Typically, a p85 isoform or a peptide thereof is used to prepare antibodies by immunizing a suitable subject, (e.g., rabbit, goat, mouse or other mammal) with the immunogen. An appropriate immunogenic preparation can contain, for example, p85 obtained by expression of the sequence encoding p85 or by gene activation, or a chemically synthesized p85 peptide. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881 which are hereby expressly incorporated by reference in their entirety. The preparation can further include an adjuvant, such as Freund's complete or incomplete adjuvant, or similar immunostimulatory agent. Immunization of a suitable subject with an immunogenic p85 preparation induces a polyclonal anti-target protein antibody response.

Anti-p85 antibodies or fragments thereof can be used as a p85 inactivating agent. Examples of anti-p85 antibody fragments include F(v), Fab, Fab′ and F(ab′)2 fragments which can be generated by treating the antibody with an enzyme such as pepsin. The term “monoclonal antibody” or “monoclonal antibody composition”, as used herein, refers to a population of antibody molecules that contain only one species of an antigen binding site capable of immunoreacting with a particular epitope of the target protein. A monoclonal antibody composition thus typically displays a single binding affinity for the particular target protein with which it immunoreacts.

Additionally, anti-p85 antibodies produced by genetic engineering methods, such as chimeric and humanized monoclonal antibodies, comprising both human and non-human portions, can be made using standard recombinant DNA techniques. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Patent 5,225,539; Jones et al., Nature 321:552-525, 1986; Verhoeyan et al., Science 239:1534, 1988; and Beidler et al., J. Immunol. 141:4053-4060, 1988.

In addition, a monoclonal antibody directed against p85 can be made using standard techniques. For example, monoclonal antibodies can be generated in transgenic mice or in immune deficient mice engrafted with antibody-producing cells, e.g., human cells. Methods of generating such mice are described, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with p85 or an antigenic p85 peptide and splenocytes from these immunized mice can then be used to create hybridomas. Methods of hybridoma production are well known.

Human monoclonal antibodies against p85 can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J 12:725-734. In addition, a combinatorial library of antibody variable regions can be generated by mutating a known human antibody. For example, a variable region of a human antibody known to bind the target protein, can be mutated, by for example using randomly altered mutagenized oligonucleotides, to generate a library of mutated variable regions which can then be screened to bind to the target protein. Methods of inducing random mutagenesis within the CDR regions of immunoglobulin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds p85. In a preferred embodiment, the primary screening of the library involves panning with the immobilized p85 and display packages expressing antibodies that bind the immobilized p85 are selected.

Display Libraries

The methods described herein can involve the use of peptides that inhibit or reduce a p85 isoform activity, to thereby treat a subject having an insulin related disorder, e.g., diabetes. A display library can be screened to identify peptides that reduce or inhibit p85.

In one approach for screening for p85 binding peptides, the candidate peptides are displayed on the surface of a cell or viral particle, and the ability of particular cells or viral particles to bind p85 via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands can be used to detect homologs that retain ligand-binding activity. The use of fluorescently labeled ligands allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH2-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having an insulin-related disorder, the method comprising reducing the amount, expression, or activity of a p85 isoform in a cell or tissue of the subject.

2. The method of claim 1, wherein reducing the amount, expression, or activity of a p85 isoform comprises administering an anti-p85 antibody.

3. The method of claim 1, wherein reducing the amount, expression, or activity of a p85 isoform comprises administering a small molecule that reduces the amount, expression, or activity of a p85 isoform.

4. The method of claim 1, wherein the insulin related disorder is diabetes; obesity; hyperglycemia; hypertension; polycystic ovarian disease; or hypolipidemia.

5. The method of claim 1, wherein the insulin related disorder is Type 2 diabetes.

6. The method of claim 1, wherein the p85 isoform is a p85α isoform.

7. The method of claim 6, wherein reducing the amount, expression, or activity of a p85α isoform comprises administering an anti-p85α antibody.

8. The method of claim 6, wherein reducing the amount, expression, or activity of a p85α isoform comprises administering a small molecule that reduces the amount, expression, or activity of a p85α isoform.

9. The method of claim 6, wherein the cell or tissue is a liver, heart, fat, or skeletal muscle cell or tissue.

10. The method of claim 1, wherein the p85 isoform is p85β.

11. The method of claim 10, wherein reducing the amount, expression, or activity of a p85β isoform comprises administering an anti-p85β antibody.

12. The method of claim 10, wherein reducing the amount, expression, or activity of a p85 β isoform comprises administering a small molecule that reduces the amount, expression, or activity of a p85β isoform.

13. The method of claim 6, further comprising reducing the amount, expression, or activity of all p85α isoforms.

14. The method of claim 13, wherein reducing the amount, expression, or activity of all p85α isoforms comprises administering an anti-p85α antibody that recognizes all p85α isoforms.

15. The method of claim 13, wherein reducing the amount, expression, or activity of all p85α isoforms comprises administering a small molecule that reduces the amount, expression, or activity of all p85α isoforms.

16. The method of claim 1, wherein the subject is an experimental animal.

17. The method of claim 1, wherein the subject is a human.

18. The method of claim 6, wherein the cell is a liver, heart, fat, or skeletal muscle cell.

19. The method of claim 10, wherein the cell or tissue is a muscle or brown adipocyte cell or tissue.

20. A method of screening for a compound for treatment of an insulin related disorder, comprising:
providing a test agent;
administering the test agent to a cell, tissue, or experimental animal; and
evaluating the ability of the test agent to reduce the activity of a p85 isoform, wherein an agent that reduces the activity of a p85 isoform is identified as an agent for the treatment of an insulin related disorder.

21. The method of claim 20, wherein the ability of the agent to reduce the activity of a p85 isoform in the cell, tissue, or experimental animal is evaluated by evaluating PI3K or p110 activity in the cell, tissue, or experimental animal.

22. The method of claim 20, wherein the ability of the agent to reduce the activity of a p85 isoform in the cell, tissue, or experimental animal is evaluated by determining the ability of the agent to affect insulin sensitivity in the cell, tissue, or experimental animal.

23. The method of claim 20, wherein the cell or tissue is a fat, liver, heart, or muscle cell or tissue.

24. The method of claim 20, wherein the experimental animal is a model of insulin resistance.

25. The method of claim 20, wherein the agent is selected from the group consisting of a peptide, an antibody and a small molecule.

26. The method of claim 20, wherein the insulin related disorder is diabetes or hyperglycemia.

27. A transgenic animal lacking expression of p85α, p55α, and p50α.

28. A transgenic animal lacking expression of p85β.

29. The transgenic animal of claim 27, wherein the transgenic animal is a model of insulin resistance.

30. The transgenic animal of claim 28, wherein the transgenic animal is a model of insulin resistance.

31. A method of identifying a subject at risk for an insulin-related disorder, the method comprising evaluating the amount, expression, or activity of a p85 isoform in a cell or tissue of a subject to determine if the subject is at risk.

32. A method of analyzing a treatment for its effect on insulin metabolism, the method comprising: a) providing a cell, tissue, or experimental animal in which the expression of p85 has been altered; b) administering the treatment to the cell, tissue, or experimental animal; and c) evaluating the effect of the treatment on insulin metabolism in the cell, tissue, or experimental animal, thereby analyzing a treatment for its effect on insulin metabolism
